(19) United States
(12) Patent Application Publication
(10) Pub. No.: US 2023/0028690 A1
(43) Pub. Date: Jan. 26, 2023

(54) METHOD AND SYSTEM OF DEPLOYING A WEARABLE TRANSDERMAL BASED VEHICLE IGNITION INTERLOCK

(71) Applicant: Consumer Safety Technology LLC, Des Moines, IA (US)

(72) Inventors: Shannon Woods, Des Moines, IA (US); Thomas Chess, Des Moines, IA (US)

(21) Appl. No.: 17/383,952

(22) Filed: Jul. 23, 2021

(51) Int. Cl. B60R 25/01; B60R 25/20; B60R 25/24; B60R 25/32; A61B 5/00; A61B 5/1477; A61B 5/18

(52) U.S. Cl. CPC B60R 25/01; B60R 25/20; B60R 25/24; B60R 25/32; A61B 5/4845; A61B 5/1477; A61B 5/18; A61B 5/002; B60R 2325/101; B60R 2325/103

(57) ABSTRACT

A system and method of deploying an ignition interlock system. The method comprises identifying a wearable transdermal device in response to initiating wireless communication between a controller device of a vehicle system and the wearable transdermal device; receiving a transdermal alcohol measurement transmitted from the wearable transdermal device; and generating, in accordance with the transdermal alcohol measurement being above a predetermined intoxication threshold, a vehicle ignition lockout state of the vehicle system.

### TECHNICAL FIELD

The disclosure herein relates to vehicle ignition interlock systems and methods of operation thereof.

### BACKGROUND

Vehicles can incorporate a breath alcohol ignition interlock device (IID) to prevent a driver from operating a vehicle while intoxicated with alcohol. Once installed and deployed in a particular vehicle, IIDs are designed to prevent a driver from starting and operating the vehicle, typically based upon the driver's breath alcohol content (BrAC) being above a mandated alcohol concentration threshold. Various jurisdictions have adopted laws providing for use of such IID devices as a sanction for drivers convicted of driving while intoxicated, or as a condition of restoring driving privileges during some probationary period after such offenses. A typical IID meets guidelines established by the National Highway Traffic Safety Administration (NHTSA) in accordance with published specifications for IIDs which delineate various tests that such the IID must pass in order to effectively and reliably pre-empt intoxicated driving.

### BRIEF DESCRIPTION OF THE DRAWINGS

FIG. 1 illustrates, in an example embodiment, a vehicle ignition interlock system incorporating a wearable transdermal device.

FIG. 2 illustrates, in one embodiment, an example architecture of a wearable transdermal device deployed within a vehicle ignition interlock system.

FIG. 3 illustrates, in an example embodiment, a method of deploying a wearable transdermal device within an ignition interlock system.

FIG. 4 illustrates, in an example embodiment, a method of deploying a wearable transdermal device within an ad hoc ignition interlock system.

### DETAILED DESCRIPTION

Embodiments herein recognize the need for continuously monitoring a driver's intoxication state, in some embodiments via measurements representative of a driver's blood alcohol content, prior to even entering a vehicle, then applying such a prior measurement while the driver attempts to start the vehicle ignition. Embodiments herein further recognize a need for a less intrusive and arguably more discreet IID deployment compared to a breath alcohol based IID that requires a vehicle driver to provide a breath sample, and very often multiple breath samples, by blowing into the mouthpiece of a separate and distinct handheld module of the IID.

Among other benefits and technical effects, embodiments herein provide a method and system of deploying, in conjunction with an ignition interlock system, continuous monitoring of an intended driver's alcohol content via transdermal alcohol sensing. The term continuous monitoring as used herein means monitoring during time durations that include prior to a driver entering a vehicle and attempting ignition start, during ignition start, and even during operation of the vehicle subsequent to a successful ignition start. Yet other benefits and technical effects are provided by a transdermal alcohol monitoring sensor of a transdermal wearable device which is not limited for operation with a particular vehicle by virtue of a hard-wired installation, such as exemplified by an IID breath alcohol handheld module. Rather, among other benefits, the transdermal wearable device is sufficiently modular for deployment in any vehicle system operationally equipped with an IID controller module and an IID relay module interconnected to an ignition system of the vehicle, and capable of being communicatively interconnected with the transdermal wearable device. In such embodiments, the transdermal wearable device can be deployed, in effect forming an ad hoc ignition interlock system, independent of a specific vehicle, based on broadcasting, by wireless radio frequency communication, its unique identity to an IID controller device of each different, specific vehicle system within which deployment is desired.

Embodiments herein further provide for reporting to third party monitoring services or state authorities in real-time during the continuous monitoring of an intended driver's intoxication or impairment state in context of vehicle ignition attempts, and even during subsequent vehicle operation.

Provided is a method of deploying an ignition interlock system. The method comprises identifying a wearable transdermal device in response to initiating wireless communication between a controller device of a vehicle system and the wearable transdermal device, receiving a transdermal alcohol measurement transmitted from the wearable transdermal device and generating, in accordance with the transdermal alcohol measurement being above a predetermined intoxication threshold, a vehicle ignition lockout state of the vehicle system.

Also provided is an ignition interlock system including a processor and a non-transitory memory storing executable instructions. The instructions, when executed in the processor, cause the processor to perform operations comprising identifying a wearable transdermal device in response to initiating wireless communication between a controller device of a vehicle system and the wearable transdermal device, receiving a transdermal alcohol measurement transmitted from the wearable transdermal device and generating, in accordance with the transdermal alcohol measurement being above a predetermined intoxication threshold, a vehicle ignition lockout state of the vehicle system.

Further provided is a non-transitory computer readable memory storing instructions executable in a processor device. The instructions when executed in the processor, cause the processor device to perform operations comprising identifying a wearable transdermal device in response to initiating wireless communication between a controller device of a vehicle system and the wearable transdermal device, receiving a transdermal alcohol measurement transmitted from the wearable transdermal device and generating, in accordance with the transdermal alcohol measurement being above a predetermined intoxication threshold, a vehicle ignition lockout state of the vehicle system.

Embodiments described herein can be implemented using programmatic modules, through the use of instructions that are executable by one or more processors. A programmatic module can include a program, a sub-routine, a portion of a program, or a software component or a hardware component capable of performing one or more stated tasks or functions. As used herein, a programmatic module can exist on a hardware component independently of other modules or components, or can be a shared element of other modules, programs or machines.

Some embodiments of ignition interlock systems described herein can generally incorporate computing and communication resources, such as processor and memory resources. Memory, processing, and network resources, local or remote but communicatively accessible, may be applied in connection with the establishment, use, or performance of any embodiment described herein, including with the performance of any method or with the implementation of any system.

One or more embodiments described herein provide that methods, techniques, and actions performed by an IID based monitoring device are performed programmatically, or as a computer-implemented method. Programmatically, as used herein, means through the use of code or computer-executable instructions. These instructions can be stored in one or more memory resources accessible to an IID based monitoring device.

### Devices and System

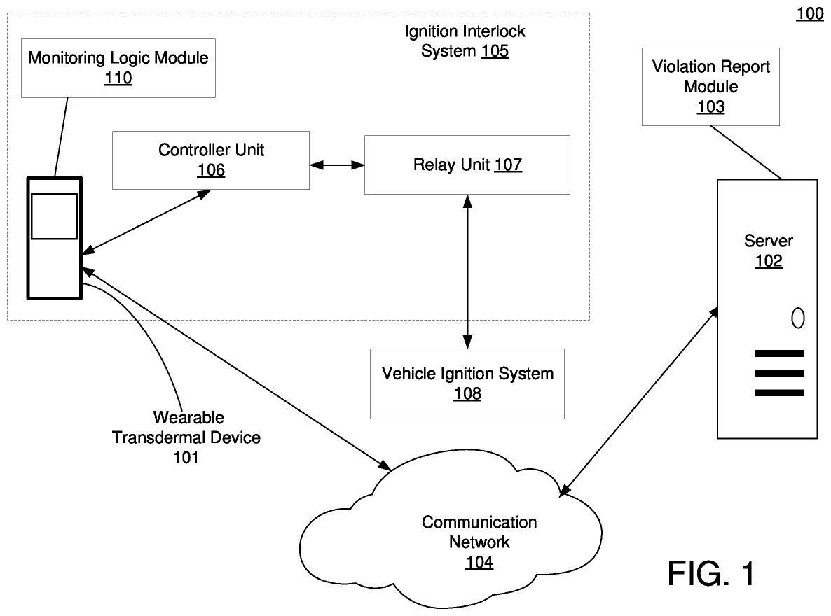

FIG. 1 illustrates, in an example embodiment, vehicle ignition interlock system 105 incorporating wearable transdermal device 101 within vehicle system 100. Vehicle ignition interlock system 105, in embodiments, includes relay unit 107 and controller unit 106 electrically and communicatively interconnected within vehicle system 100. Relay unit 107, in embodiments, enables vehicle ignition system 108 to transition from ignition on and ignition off states, wherein a vehicle can be rendered in operational or non-operational states respectively. Wearable transdermal device 101, in embodiments, includes a processor and a memory, and also comprises a transdermal alcohol sensor membrane device configured to measure an alcohol content based at least partly upon contact with a source of transdermal perspiration, such as from a bearer's skin surface. The memory of wearable transdermal device 101, in embodiments, stores executable instructions constituting monitoring logic module 110 for deploying wearable transdermal device 101 within ignition interlock system 100, and also stores IID and driver account details, including driver identification and history of usage of the IID associated with the particular driver and driver account.

Wearable transdermal device 101, in embodiments, is communicatively interconnected with violation report module 103 of server 102 via communication network 104. In some variations, the executable instructions constituting monitoring logic module 110, or portions thereof, can be hosted at server 102 which can be communicatively accessible to wearable transdermal device 101 via communication network 104. Violation report module 103 of server 102 includes logic related to storing IID and driver account details, including driver identification and authentication features in conjunction with history of usage of the IID associated with a particular driver and driver account. As referred to variously herein, a driver or vehicle driver can be the wearer, or bearer, of wearable transdermal device 101. In some variations, violation report module 103 of server 102 can be managed and maintained in deployment by an IID services provider, and allows reporting of driver and related IID violations to state authorities. In some embodiments herein, alcohol content measurements from wearable transdermal device 101 can be correlated with blood alcohol content (BAC) levels, and a violation report can also be triggered based on either of a measured BAC or a correlated BAC. It is contemplated that such BAC correlations may be performed either locally at wearable transdermal device 101 or at server 102, either in real time or subsequently established.

In some embodiments of ignition interlock system 105 as depicted in FIG. 1, wearable transdermal device 101 can be communicatively interfaced with controller unit 106 and relay unit 107. Relay unit 107 can directly interface with vehicle engine ignition system 108 in order to operationally disable the vehicle ignition system in response to transdermal alcohol measurements, as measured using one or more transdermal alcohol sensor devices of wearable transdermal device 101, being above a proscribed or mandated threshold level. Although controller unit 106 and relay unit 107 are depicted in FIG. 1 as separate modules, it is contemplated that some or all functionality of the modules thereof can be integrated and physically incorporated rather than the separate modules as depicted. Furthermore the communication between one or more modules may be deployed using physically wired connections, wireless connections such as, but not necessarily limited to, BLUETOOTH, WiFI, a telephony-based cellular communication protocol and any combination of wired and wireless communications. In one embodiment, ignition interlock system 105 can be communicatively interfaced with the vehicle engine system via an on-board diagnostic (OBD) port using a wireless communication protocol such as, but not necessarily limited to, BLUETOOTH.

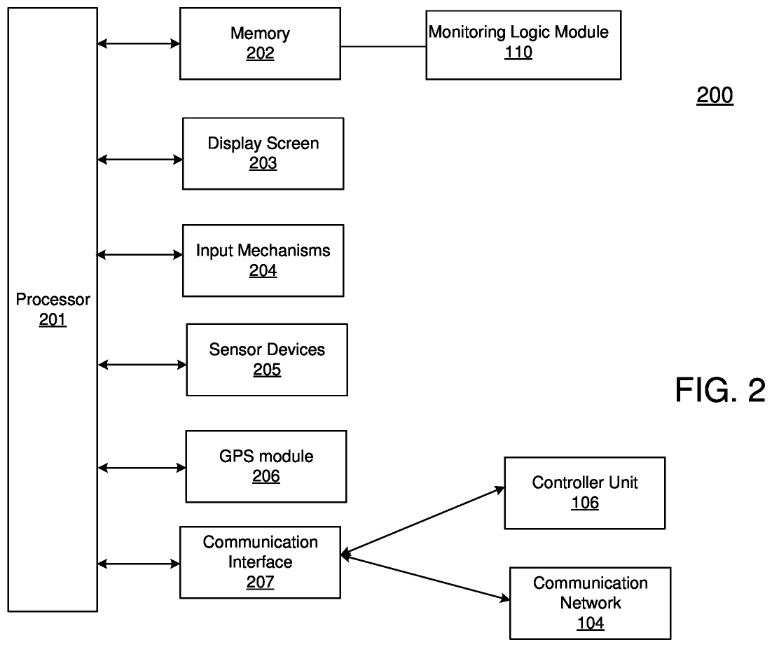

FIG. 2 illustrates, in one embodiment, example architecture 200 of wearable transdermal device 101 as deployed within ignition interlock system 105. In one embodiment, wearable transdermal device 101 can include processor 201, memory 202, display screen 203, input mechanisms 204 such as resistive- or capacitance-based input mechanisms or software-implemented touchscreen input functionality, audible alerts capability, and sensor devices 205 including one or more transdermal alcohol sensors. Wearable transdermal device 101 may also include capability for detecting and communicatively accessing wireless communication signals, including but not limited to any of BLUETOOTH, Wi-Fi, RFID, and global positioning system (GPS) signals, and incorporate communication interface 207 for communicatively coupling to communication network 104, such as by sending and receiving cellular and GPS data over data channels. Communication interface 207 of wearable transdermal device 101 can enable wireless communication via wireless communication protocols including, but not limited to, BLUETOOTH, Wi-Fi, RFID and near field communication (NFC), with controller unit 106. Wearable transdermal device 101 can also incorporate GPS module 206 that includes GPS receiver and transmitter circuitry for accessing and enabling GPS signals and data.

Sensor devices 205 of wearable transdermal device 101 can include one or more transdermal alcohol sensors that measures alcohol content inherent in the driver's perspiration, thereby providing at least an objective indication of the blood alcohol concentration in the driver's bloodstream. The transdermal alcohol sensor generates an electrical signal generally proportionate to alcohol content in accordance with the transdermal measurements. If the vehicle operator or intended driver's transdermal alcohol content does not exceed the threshold, the operator is determined as not intoxicated or impaired. If wearable transdermal device 101 registers a higher alcohol content than a predetermined allowable threshold, which in some embodiments can range from 0.02% through 0.08% alcohol content, wearable transdermal device 101 generates, records and reports an impaired driving violation. Sensor devices 205 can further include digital imaging sensors for incorporating digital imaging or camera capability at wearable transdermal device 101.

Monitoring logic module 110 can be constituted of computer processor-executable code stored in memory 202 of wearable transdermal device 101 for deploying functionality ascribed to embodiments herein. In some embodiments, monitoring logic module 110 may be stored in memory 202 upon subsequent accessing and downloading, via communication network 104, from server 102 or other third party remote server.

In particular embodiments, monitoring logic module 110 includes instructions executable in processor 201 to identify wearable transdermal device 101 in response to initiating wireless communication between controller unit 106 of vehicle system 100 and wearable transdermal device 101. In embodiments, prior to initiating the wireless communication, the transdermal alcohol measurement that indicates an alcohol impairment or intoxication state can be sensed and established by way of continual monitoring even before an operator attempts to operate or drive the vehicle. Wearable transdermal device 101 borne by the intended vehicle driver or operator may be any of a wrist wearable, arm wearable or an ankle wearable bracelets, or a transdermal patch applied to, or wearable on, the skin surface of a body portion or member including on or around the chest portion or as a necklace. Wearable transdermal device 101 can incorporate one or more transdermal alcohol sensor membrane devices configured to detect and measure an alcohol content based at least partly upon contact with a source of transdermal perspiration.

In some aspects, identifying the wearable transdermal device can be performed in response to initiating the wireless communication in accordance with a given wireless communication protocol, including, though not limited to, BLUETOOTH, WiFI and a near field communication (NFC) protocol.

Monitoring logic module 110 also includes instructions executable in processor 201 to receive a transdermal alcohol measurement transmitted from wearable transdermal device 101 in accordance with the given communication protocol.

Monitoring logic module 110 further includes instructions executable in processor 201 to generate, in accordance with the transdermal alcohol measurement being above a predetermined intoxication threshold, a vehicle ignition lockout state of vehicle system 100. In some embodiments, prior to the generating, one or more digital images of the intended driver may be taken in authentication of the intended driver in conjunction with an IID account associated with wearable transdermal device 101. The predetermined intoxication threshold may be applied as a proxy for a blood alcohol content in a range from 0.02% to 0.08%, though higher and lower values than that particular range may be deployed.

In embodiments, monitoring logic module 110 further includes executable instructions enabling processor 201 to perform operations comprising reporting, to a central monitoring server or server 102, ignition interlock system or IID account information uniquely associated with the vehicle driver that bears transdermal wearable device 101, in conjunction with event timestamp information. Based on continuous monitoring via wearable transdermal device 101, an ongoing series of transdermal measurements, in embodiments, can indicate transdermal alcohol content measurements taken over any continuous 24 hour period, prior to, as well as after, the driver bearing wearable transdermal wearable device 101 enters a vehicle with intention to drive. Accumulated periodic measurements of transdermal alcohol content may include values being above and also below the predetermined alcohol threshold. In some variations, the event timestamped information an comprise vehicle dynamic event data acquired in accordance with one or more vehicle sensors of the vehicle system while the vehicle is being driven. The vehicle sensors may detect vehicle driving dynamics data reflective of the manner in which the vehicle is being driven, using vehicle sensors including, but not limited to, one or more accelerometers, gyroscope and compass.

### Methodology

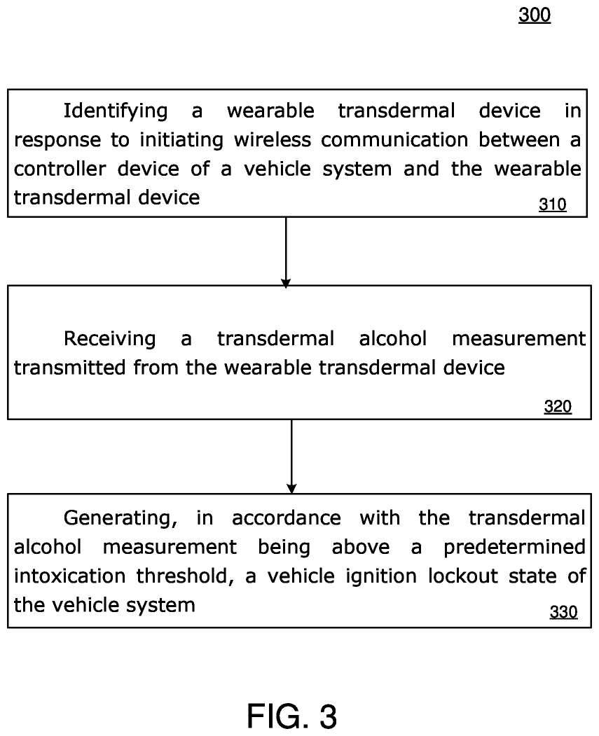

FIG. 3 illustrates, in an example embodiment, method 300 of deploying wearable transdermal device 101 within ignition interlock system 105. Examples of method steps described herein are related to deployment and use of wearable transdermal device 101 as described herein. According to one embodiment, the techniques are performed in processor 201 executing one or more sequences of software logic instructions that constitute monitoring logic module 110 of wearable transdermal device 101. In some variations, at least portions of monitoring logic module 110 may be hosted at, or remotely sourced from, violation report server 102 (also referred to herein as server 102) that is communicatively coupled with wearable transdermal device 101. Such instructions may be read into memory 202 from machine-readable medium, such as memory storage devices. Executing the instructions of monitoring logic module 110 stored in memory 202 causes processor 201 to perform the process steps described herein. In alternative implementations, at least some hard-wired circuitry may be used in place of, or in combination with, the software logic instructions to implement examples described herein. Thus, the examples described herein are not limited to any particular combination of hardware circuitry and software instructions.

At step 310, identifying wearable transdermal device 101 in response to initiating wireless communication between controller unit 106 of ignition interlock system 105 and wearable transdermal device 101. In embodiments, prior to initiating the wireless communication, the transdermal alcohol measurement that indicates an alcohol impairment or intoxication state may be sensed and established by way of continual monitoring even before an operator attempts to drive the vehicle. Wearable transdermal device 101 borne by the intended vehicle driver or operator may be wrist wearable, arm wearable or an ankle wearable bracelets, or a transdermal patch applied to, or wearable on, the skin surface of a body portion or limb of the bearer. Wearable transdermal device 101 can incorporate one or more transdermal alcohol sensor membrane devices configured to detect and measure an alcohol content based at least partly upon contact with a source of transdermal perspiration.

In some aspects, identifying the wearable transdermal device can be performed in response to initiating the wireless communication in accordance with a given wireless communication protocol, including, though not limited to, BLUETOOTH, WiFI and a near field communication (NFC) protocol.

At step 320, receiving a transdermal alcohol measurement transmitted from wearable transdermal device 101.

At step 330, generating, in accordance with the transdermal alcohol measurement being above a predetermined intoxication threshold, a vehicle ignition lockout state of ignition interlock system 105. In some embodiments, prior to the generating, one or more digital images of the intended driver may be taken in authentication of the intended driver in conjunction with an IID account associated with wearable transdermal device 101. The predetermined intoxication threshold may be applied as a proxy for a blood alcohol content in a range from 0.02% to 0.08%, though higher and lower values than that particular range may be deployed.

In another aspect, the method further comprises reporting, to a central monitoring server or server 102, ignition interlock system account information uniquely associated with the vehicle driver that bears transdermal wearable device 101, in conjunction with event timestamp information, a series of transdermal alcohol measurements. The series, in some embodiments, can indicate transdermal alcohol content measurements taken over any continuous 24 hour period, prior to as well as after the driver bearing wearable transdermal wearable device 101 enters a vehicle with intention to drive, and accumulating periodic measurements of transdermal alcohol content, where such measurement may include values being above and also below the predetermined alcohol threshold. In some variations, the event timestamped information comprises vehicle dynamic event data acquired in accordance with one or more vehicle sensors of the vehicle system while the vehicle is being driven. The vehicle sensors may detect vehicle driving dynamics data reflective of the manner in which the vehicle is being driven, using vehicle sensors including, but not limited to, one or more accelerometers, gyroscope and compass.

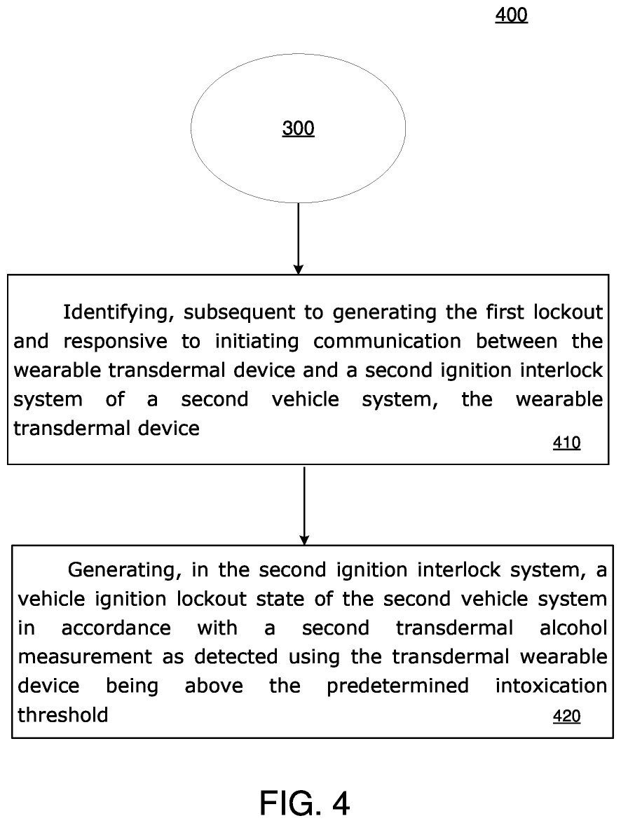

FIG. 4 illustrates, in an example embodiment, method 400 of deploying wearable transdermal device 101 within ad hoc ignition interlock systems formed by virtue of sequential or serial deployment across one or more different vehicles that include respective controller units that are communicatively interfaceable with wearable transdermal device 101. The communicative interfacing can, in some embodiments, be triggered in response to wearable transdermal device 101 being detected, for example via NFC communication and proximity sensing techniques, within a predetermined proximity of controller unit 106 of any vehicle system. In some variations, the communicative interfacing can, in some embodiments, be triggered in response to wearable transdermal device 101 being detected, for example via NFC communication and proximity sensing techniques, within a driver's seat, or a predetermined proximity of the driver's seat, of a given vehicle. Deployment of the ad hoc ignition interlock system as described herein can commence, in some examples, upon successful wireless communication pairing of wearable transdermal device 101 with a controller unit of any given vehicle or vehicle system based on proximity detection considerations.

In some embodiments, a wearer of the wearable transdermal device may be detected in the passenger seat, rather than in the driver's seat of the vehicle. Such detection can be based on proximity of the wearable in conjunction with one or more images acquired in real time from the camera system detecting or authenticating an occupant other than the person bearing the wearable transdermal device being in the driver's seat of the vehicle. In such embodiments, the ignition interlock system could selectively be disengaged from triggering, allowing the non-bearer driver to start the vehicle and drive even when the wearable transdermal device records and transmits an alcohol impairment level or BAC that is above an applicable predetermined intoxication threshold.

The illustrative techniques of FIG. 4, in some embodiments, can be combined with one or more of the example embodiment steps as described in regard to FIG. 3.

At step 410, identifying, subsequent to generating the first lockout and responsive to initiating communication between wearable transdermal device 101 and a second ignition interlock system of a second vehicle system, wearable transdermal device 101 as being uniquely associated with a bearer of wearable transdermal device 101.

At step 420, generating, in the second ignition interlock system, a vehicle ignition lockout state of the second vehicle system in accordance with a second transdermal alcohol measurement as detected using transdermal wearable device 101 being above the predetermined intoxication threshold.

It is contemplated for embodiments described herein to extend to individual elements and concepts described herein, independently of other concepts, ideas or system, as well as for embodiments to include combinations of elements recited anywhere in this application. For example, although techniques of monitoring described herein are disclosed in context of transdermal alcohol sensor devices and alcohol impaired driving, it is contemplated that such techniques, in conjunction with appropriate, respective transdermal intoxicant sensing devices, be applied to alternate intoxicant agents that result in impaired driving. Such intoxicants can include, but not necessarily be limited to, drugs including cannabinoids, opiates, and methamphetamines.

Although embodiments are described in detail herein with reference to the accompanying drawings, it is contemplated that the disclosure herein is not limited to only such literal embodiments. As such, many modifications including variations in sequence of the method steps in conjunction with varying combinations of user interface features disclosed herein will be apparent to practitioners skilled in this art. Accordingly, it is intended that the scope of the invention be defined by the following claims and their equivalents. Furthermore, it is contemplated that a particular feature described either individually or as part of an embodiment can be combined with other individually described features, or parts of other embodiments. Thus, the absence of describing combinations of such do not preclude the inventor from claiming rights to such combinations.

What is claimed is:

1. A method of deploying an ignition interlock system, the method comprising:
identifying a wearable transdermal device in response to initiating wireless communication between a controller device of a vehicle system and the wearable transdermal device;
receiving a transdermal alcohol measurement transmitted from the wearable transdermal device; and
generating, in accordance with the transdermal alcohol measurement being above a predetermined intoxication threshold, a vehicle ignition lockout state of the vehicle system.

2. The method of claim 1 further comprising, prior to the initiating, establishing the transdermal alcohol measurement, the transdermal alcohol measurement being representative of an alcohol intoxication state of a user bearing the wearable transdermal device.

3. The method of claim 2 further comprising, prior to the generating, receiving at least one digital image in authentication of the user.

4. The method of claim 1 further comprising identifying the wearable transdermal device in response to initiating the wireless communication in accordance with at least one of a BLUETOOTH, a WiFI, a near field communication (NFC) protocol and a cellular communication protocol.

5. The method of claim 1 wherein the wearable transdermal device comprises a transdermal alcohol sensor membrane device configured to measure an alcohol content based at least partly upon contact with a source of transdermal perspiration.

6. The method of claim 5 wherein the wearable transdermal device comprises at least one of a wrist wearable, an arm wearable, a necklace, a chest wearable and an ankle wearable bracelets, and a transdermal patch.

7. The method of claim 6 wherein the predetermined intoxication threshold comprises a proxy for a blood alcohol content in a range from 0.02% to 0.08%.

8. The method of claim 1 wherein the ignition interlock system is a first ignition interlock system of a first vehicle system, and further comprising:
identifying, subsequent to generating the first lockout and responsive to initiating communication between the wearable transdermal device and a second ignition interlock system of a second vehicle system, the wearable transdermal device; and
generating, in the second ignition interlock system, a vehicle ignition lockout state of the second vehicle system in accordance with a second transdermal alcohol measurement as detected using the transdermal wearable device being above the predetermined intoxication threshold.

9. The method of claim 1 further comprising reporting, to a central monitoring server computing device, ignition interlock system account information associated with the transdermal wearable device in conjunction with event timestamp information, a series of transdermal alcohol measurements, at least a portion of the series being one of above and below the predetermined alcohol threshold.

10. The method of claim 9 wherein the event timestamp information comprises data acquired in accordance with one or more vehicle sensors of the vehicle system, the one or more vehicle sensors comprising at least one of an accelerometer, a gyroscope and a compass.

11. An ignition interlock system comprising:
a processor; and
a non-transitory memory including instructions, the instructions when executed causing the processor to perform operations comprising:
identifying a wearable transdermal device in response to initiating wireless communication between a controller device of a vehicle system and the wearable transdermal device;
receiving a transdermal alcohol measurement transmitted from the wearable transdermal device; and
generating, in accordance with the transdermal alcohol measurement being above a predetermined intoxication threshold, a vehicle ignition lockout state of the vehicle system.

12. The ignition interlock system of claim 11 further comprising instructions executable to, prior to the initiating, establish the transdermal alcohol measurement, the transdermal alcohol measurement being representative of an alcohol intoxication state of a user bearing the wearable transdermal device.

13. The ignition interlock system of claim 12 further comprising instructions executable to, prior to the generating, receive at least one digital image in authentication of the user.

14. The ignition interlock system of claim 11 further comprising instructions executable to identify the wearable transdermal device in response to initiating the wireless communication in accordance with at least one of a BLUETOOTH, a WiFI, a near field communication (NFC) protocol and a cellular communication protocol.

15. The ignition interlock system of claim 11 wherein the wearable transdermal device comprises a transdermal alcohol sensor membrane device configured to measure an alcohol content based at least partly upon contact with a source of transdermal perspiration.

16. The ignition interlock system of claim 15 wherein the wearable transdermal device comprises at least one of a wrist wearable, an arm wearable, a necklace, a chest wearable and an ankle wearable bracelets, and a transdermal patch.

17. The ignition interlock system of claim 16 wherein the predetermined intoxication threshold comprises a proxy for a blood alcohol content in a range from 0.02% to 0.08%.

18. The ignition interlock system of claim 11 wherein the ignition interlock system is a first ignition interlock system of a first vehicle system, and further comprising instructions executable to:
identify, subsequent to generating the first lockout and responsive to initiating communication between the wearable transdermal device and a second IID of a second vehicle system, the wearable transdermal device; and
generate, in the second ignition interlock system, a vehicle ignition lockout state of the second vehicle system in accordance with a second transdermal alcohol measurement as detected using the transdermal wearable device being above the predetermined intoxication threshold.

19. The ignition interlock system of claim 11 further comprising instructions executable to report, to a central monitoring server computing device, ignition interlock system account information associated with the transdermal wearable device in conjunction with event timestamp information, a series of transdermal alcohol measurements, at least a portion of the series being one of above and below the predetermined alcohol threshold.

20. A non-transitory computer readable memory storing instructions, the instructions when executed in a processor device causing the processor device to perform operations comprising:
identifying a wearable transdermal device in response to initiating wireless communication between a controller device of a vehicle system and the wearable transdermal device;
receiving a transdermal alcohol measurement transmitted from the wearable transdermal device; and
generating, in accordance with the transdermal alcohol measurement being above a predetermined intoxication threshold, a vehicle ignition lockout state of the vehicle system.
